(19) United States
(12) Patent Application Publication
(10) Pub. No.: US 2025/0090085 A1
(43) Pub. Date: Mar. 20, 2025

(54) SYSTEM, DEVICE AND METHOD OF DETECTION AND CLASSIFICATION OF EARLY-STAGE PRESSURE INJURIES

(71) Applicant: I.R Med Ltd., Rosh Pinna (IL)

(72) Inventors: Oded BASHAN, Tel-Aviv (IL); Aharon BINUR, Kibutz Amir (IL); Ronnie KLEIN, Haifa (IL); Yaniv COHEN, Maale Adomim (IL); Roy GOVRIN, Kyriat Tivon (IL); Avi VACHNIS, Giv'at Avni (IL)

(73) Assignee: I.R Med Ltd., Rosh Pinna (IL)

(21) Appl. No.: 18/468,911

(22) Filed: Sep. 18, 2023

(51) Int. Cl. A61B 5/00; A61B 5/0205; A61B 5/1455; G06N 20/00; G16H 50/70

(52) U.S. Cl. CPC A61B 5/445; A61B 5/0082; A61B 5/02055; A61B 5/1455; A61B 5/7257; A61B 5/7267; G06N 20/00; G16H 50/70; A61B 2562/0238

(57) ABSTRACT

Device, system and method of non-invasive determination of a tissue injury, including: receiving, by at least two optical sensors, intensity and signal distance information from light reflected from the tissue over at least one point of the patient's skin, wherein the signal distance information is measured between at least one light source and the at least two optical sensors, receiving, by at least one physiological sensor, a physiological characteristic information of the tissue over at least one point of the patient's skin, training a machine learning (ML) algorithm to determine a tissue injury, and applying the ML algorithm on the received intensity and signal distance information and the received physiological characteristic information to determine a subcutaneous tissue injury in which liquids accumulate subcutaneously, in accordance with a calculated change in the received signal, the signal distance information, and the physiological characteristic information.

### FIELD OF THE INVENTION

The present invention relates to medical devices. More specifically, the present invention relates to systems, devices and methods for detecting and identifying early-stage pressure injuries, ulcers, and inflammatory wounds.

### BACKGROUND

Pressure injuries (PIs), also known as pressure ulcers or bedsores, are localized areas of damage to the skin and underlying tissues caused by prolonged pressure. PIs affect millions of people worldwide each year and are an ever-growing burden on U.S. and Western world healthcare systems. Many people die each year, including in the United States, directly from pressure injury related complications. The elderly and patients with disabilities and/or other background diseases suffer especially from an increased number of skin injuries including PIs, diabetic foot ulcers, and other skin disorders.

A possible way to reduce the costs of PI treatment is by early detection of PIs to enable proper treatment and achieve quick recovery and prevent deterioration of the injury. Wound treatment by accurate diagnosis, early detection and treatment monitoring is a key to providing adequate healthcare services.

PIs can occur in living tissue, and include stage 1 PIs (where the skin appears reddened and does not blanch) and deep-tissue pressure injuries.

The two main layers of the skin are the epidermis and the dermis, and the layer underneath is the subcutaneous layer. The mammalian skin includes different layers located one above the other from the upper stratum corneum layer to the epidermis, through the dermis and down to the subcutaneous layer containing fat and muscle tissue and bone. The entangled blood vessels in the different skin layers change in diameter, in structure and in thickness: whereas the larger diameter and thicker blood vessels are located deeper in the dermis, smaller in diameter and thinner blood vessels are located in the upper layers of the skin.

By assessing the damage in the blood vessels according to their position in different skin layers it is possible to detect location of an incipient skin tissue injury in different layers of the skin and allow higher accuracy classification of stage 1 PI (in upper layers) compared to deep tissue injury (DTI) (i.e., in the lower layers of the skin). The formation mechanism of stage 1 PI and DTI are different, whereas PI starts from top skin layers and penetrates inward, DTI starts from the deeper skin layers and expands to the top layers.

Current methods of pressure injury detection are often unreliable or invasive. For example, visual inspection is subjective and can be difficult to perform in patients with dark skin. Biopsy is a more accurate method for detection, but it is invasive and can be painful.

Some solutions of care for diagnosis and detection of PI can include a non-blanching test, which is a visual and tactile test, performed by the care giver, after identifying an edema in a body area that is prone to PIs. Such tests have several limitations, such as that the test is not based on measurement of accurate physical changes in tissue and accordingly relies on the experience and expertise of the caregiver in identifying PIs as a subjective biased measurement. An additional limitation can be that early stage deep-tissue and stage 1 Pis without any skin color change, cannot be identified by the care giver.

Early-stage Pis can occur when pressure is applied on a skin tissue, and some biological and bio-chemical processes start to take place in a form of tissue inflammatory reaction to the applied pressure. During the inflammatory reaction, the skin tissue changes following applied pressure that can occur both at the cellular level and in the tissue level of the skin in different skin layers. Conditions such as hypoxia, ischemia and necrosis both in the cellular and tissue level take place, because of reduced blood supply the pressed-tissue and breaks down of cells membranes due to mechanical pressure.

Tissue changes following pressure exposure, or during tissue regeneration processes, and changes in skin structure, can be monitored and followed by optical means, for example tissue light absorption and tissue light reflection changes during development of a tissue injury (development of a pressure injury as a diabetic foot ulcer, a venous ulcer etc.). Since mechanical pressure inflicts structural change in tissue, physical structure, causes biochemical processes that may lead at the end of the cell and tissue reaction to cells death, skin tissue death and cell skin decomposition, blood vessel blockage, blood vessels increased porosity, fluid leakage through blood vessel wall and disintegration.

These processes alter additional tissue properties including at least one of: tissue temperature, tissue stiffness, tissue hydration, tissue blood concentration, tissue blood flow velocity, arterial hemodynamic flow, capillary hemodynamic flow, endothelial hemodynamic flow, tissue capillary blood flow rate, tissue conductivity of heart pulse wave (tissue pulse wave velocity), other heart rate pulse conductive parameters, tissue sound wave conductivity, tissue layer structure, and changes of the normal tissue structure, e.g. collagen formation.

For example, measuring skin ion capacitance or skin electrical conductivity can be used to detect subdermal liquid accumulation as a possible indicator of a pressure injury. Measurement of temperature (e.g., by thermal infrared imaging of suspected skin injuries to map superficial temperature of the skin, creating an artificial thermal image or map of skin temperature) may be used to identify blood flow in the skin.

Pulse Wave Velocity (PWV) is another parameter that may be used to estimate tissue and blood vessel integrity by monitoring change in speed of a pulse wave spreading through the tissue blood vessels. Other heart pulse wave parameters, such as pulse amplitude, pulse shape, and pulse intensity changes, correspond with tissue damage due to prolonged pressure.

An early stage of PI can be detected by optical means because it affects skin properties. Superficial pressure ulcers can affect mainly the thin blood vessels of the dermis while the most serious pressure damage can occur as a result of deformations in the deep tissues where pressures can be much higher than at the skin interface, and where larger blood vessels are more likely to be affected, leading to necrosis of a large volume of tissue. The main difference between the two kinds of PI (the superficial and the deep tissue injury (DTI)) is the onset layer. While the superficial PI can affect mainly the dermis, the DTI can begin in the muscle and subcutaneous tissues and deteriorate this tissue first. In the next stage, the supporting structures of the skin are destroyed, and parts of the skin become nonviable and die subsequent to the primary deep tissue destruction.

To distinguish between the two kinds of PI there is a need to separate between the damaged dermis tissue and subcutaneous tissue. That may be done by optimizing the detection sensitivity in term of: maximum detection contrast-maximum optical sensitivity of specific layers (dermis/subcutaneous) to optical changes versus background and/or maximum sensitivity to the change of the concentration in the chromophores involved in PI.

The major chromophores important to analyze in pressure ulcers include: skin collagen elastin typical structure, oxy-hemoglobin, deoxy-hemoglobin, total hemoglobin water, and blood concentration and blood flow regimes. The oxy/deoxy-hemoglobin changes may be due to ischemia and the water accumulation due to onset of edema in the PI region.

There is therefore a need for a more reliable and non-invasive method of pressure injury detection.

### SUMMARY OF THE INVENTION

A non-invasive method, system and device are provided for detecting and classifying early-stage PIs, diabetic foot ulcer and other skin conditions.

There is thus provided, in accordance with some embodiments of the invention, a method of non-invasive determination of a tissue injury of a patient's tissue, the method including: receiving, by at least two optical sensors, intensity and signal distance information from light reflected from the tissue over at least one point of the patient's skin, including signal distance information measured between at least one light source and the at least two optical sensors, receiving, by at least one physiological sensor over at least one point of the patient's skin, physiological characteristic information of the tissue, training, by a processor, a machine learning (ML) algorithm to determine a tissue injury, wherein the training is carried out on a dataset of intensity and signal distance information and physiological characteristic information of the tissue obtained from the sensors, applying, by the processor, the ML algorithm on the received intensity and signal distance information and the received physiological characteristic information to determine a subcutaneous tissue injury in which liquids accumulate subcutaneously, in accordance with a calculated change in the received signal intensity and signal distance information, and the physiological characteristic information, selecting, by the processor, at least one point of the patient's skin determined as a potential subdermal injury, and issuing, by the processor, an alert when a subcutaneous tissue injury is determined.

In some embodiments, a non-invasive device is attached to a mobile computing device, wherein the non-invasive device comprises the at least two optical sensors and the at least one physiological sensor, and wherein the mobile computing device comprises the processor. In some embodiments, at least one other than the point determined as a potential subdermal injury is determined as healthy tissue by the processor.

In some embodiments, modulated lighting is applied so as to accelerate the measurement time by the at least two optical sensors. In some embodiments, the modulated lighting comprises using a different frequency for different light sources simultaneously based on a Fast Fourier Transform algorithm. In some embodiments, the signal is received from the at least two optical sensors in a plurality of wavelengths.

In some embodiments, the physiological characteristic information of the tissue is selected from the group consisting of: blood flow pattern, blood flow rate, blood viscosity, tissue temperature, skin tissue capacitance, pulse wave velocity, skin elasticity, hemoglobin level, and spatial oxygenation. In some embodiments, the physiological characteristic information is determined based on detection of myoglobin in the tissue. In some embodiments, a measurement is initiated when pressure signal, from a pressure sensor, is within a predefined pressure threshold range.

In some embodiments, the pressure sensor is accommodated in an elastomeric ring. In some embodiments, a reference point is measured on a healthy tissue of the patient, in order to get a normalized personalized result for the patient.

There is thus provided, in accordance with some embodiments of the invention, a system for non-invasive determination of a tissue injury, of a patient's tissue, the system including: a light source, at least two optical sensors, configured to receive intensity and signal distance information from light reflected from the tissue over at least one point of the patient's skin, including signal distance information measured between the light source and the at least two optical sensors, at least one physiological sensor, configured to receive over at least one point of the patient's skin physiological characteristic information of the tissue, a processor, coupled to the at least two optical sensors and the at least one physiological sensor, wherein the processor is configured to: train a machine learning (ML) algorithm to determine a tissue injury, wherein the training is carried out on a dataset of intensity and signal distance information and physiological information of the tissue obtained from the sensors, apply the ML algorithm on the intensity and signal distance information obtained from the optical sensors to determine a subcutaneous tissue injury in which liquids accumulate subcutaneously, in accordance with a calculated change in the received signal intensity and signal distance information, and the physiological characteristic information, and display the determination, select at least one point of the patient's skin determined as a potential subdermal injury, and issue an alert when a subcutaneous tissue injury is determined.

In some embodiments, a signal is received from the at least two optical sensors in a plurality of wavelengths. In some embodiments, the physiological characteristic information of the tissue is selected from the group consisting of: blood flow pattern, blood flow rate, blood viscosity, temperature, skin tissue capacitance, pulse wave velocity, skin elasticity, hemoglobin level in tissue, hemoglobin saturation rate, and spatial oxygenation level. In some embodiments, the physiological characteristic information is determined based on detection of myoglobin in the tissue.

In some embodiments, the processor is to initiate a measurement when pressure signal, from a pressure sensor, is within a predefined pressure threshold range. In some embodiments, the processor is to initiate a measurement when pressure signal, from a damping pressure mechanism, is within a predefined pressure threshold range. In some embodiments, the at least two optical sensors are to measure a reference point on a healthy tissue of the patient, in order to get a normalized personalized result for the patient.

There is thus provided, in accordance with some embodiments of the invention, a device for non-invasive determination of a tissue injury of a patient's tissue, the device including: a light source, at least two optical sensors, configured to receive intensity and signal distance information from light reflected from the tissue over at least one point of the patient's skin, including signal distance information measured between the light source and the at least two optical sensors, at least one physiological sensor, configured to receive over at least one point of the patient's skin physiological characteristic information of the tissue, a processor, coupled to the at least two optical sensors and the at least one physiological sensor, wherein the processor is configured to: train a machine learning (ML) algorithm to determine a tissue injury, wherein the training is carried out on a dataset of intensity and signal distance information and physiological information of the tissue obtained from the sensors, apply the ML algorithm on the intensity and signal distance information and physiological information obtained from the optical sensors to determine a subcutaneous tissue injury in which liquids accumulate subcutaneously, in accordance with a calculated change in the received signal intensity and signal distance information, and the physiological characteristic information, select at least one point of the patient's skin determined as a potential subdermal injury, and issue an alert when a subcutaneous tissue injury is determined.

In some embodiments, the device is attachable to a mobile computing device, and wherein the internal processor sends instructions to a processor of the mobile computing device.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a block diagram of an exemplary computing device, according to some embodiments of the invention;

FIG. 2 is an illustration of a non-invasive device for determination of a tissue injury, according to some embodiments of the invention;

FIG. 3 shows an illustration of a non-invasive system for determination of a tissue injury, according to some embodiments of the invention;

FIG. 4 shows an illustration of a non-invasive system for determination of a tissue injury with a machine learning algorithm, according to some embodiments of the invention; and

FIG. 5 shows a flowchart of a method of non-invasive determination of a tissue injury, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof may occur or be performed simultaneously, at the same point in time, or concurrently.

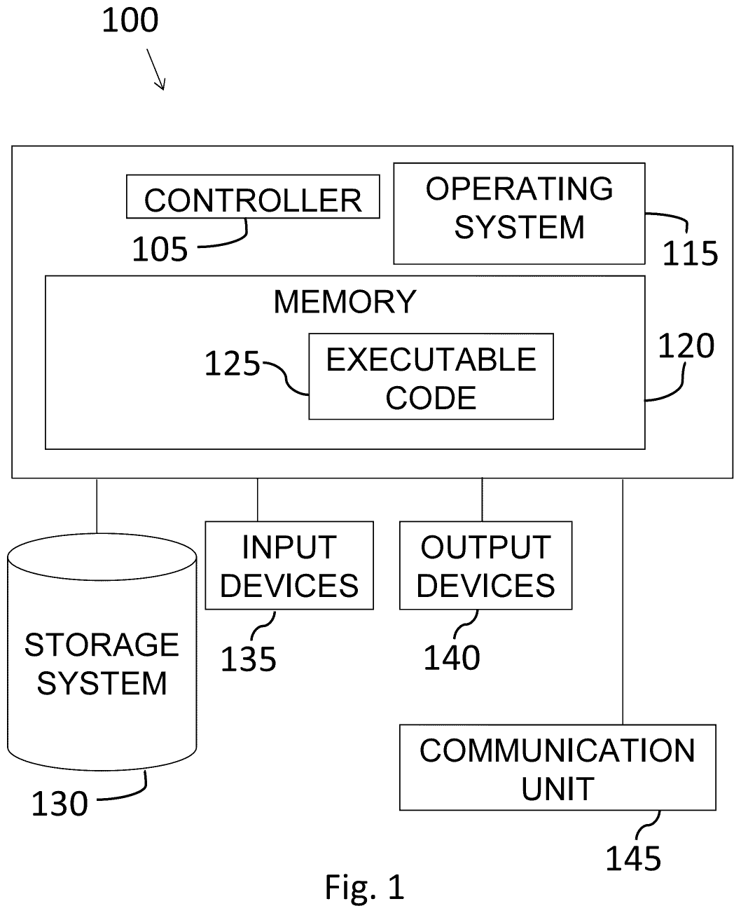

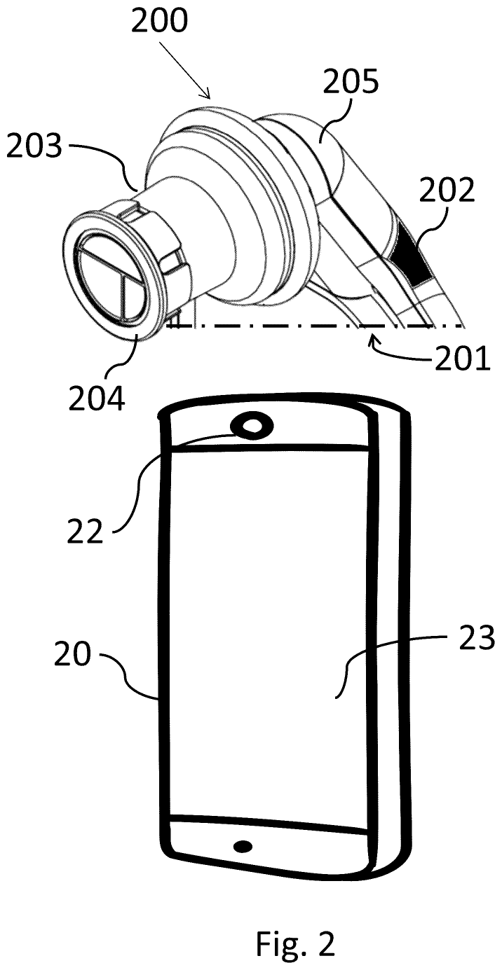

Reference is made to FIG. 1, which is a schematic block diagram of an example computing device, according to some embodiments of the invention. Computing device 100 may include a controller or processor 105 (e.g., a central processing unit processor (CPU), a chip or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet. Controller 105 may be configured to execute program code to perform operations described herein. The system described herein may include one or more computing device(s) 100, for example, to act as the various devices or the components shown in FIG. 2. For example, components of system 200 may be, or may include computing device 100 or components thereof.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of similar and/or different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein. Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 may be omitted. For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105. Such a non-transitory computer readable medium may be for example a memory, a disk drive, or a USB flash memory, encoding, including or storing instructions, e.g., computer-executable instructions, which when executed by a processor or controller, carry out methods disclosed herein. The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPUs), a plurality of graphics processing units (GPUs), or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, or any other suitable computing device. For example, a system as described herein may include one or more facility computing device 100 and one or more remote server computers in active communication with one or more facility computing device 100 such as computing device 100, and in active communication with one or more portable or mobile devices such as smartphones, tablets and the like.

According to some embodiments, a non-invasive device, system and method are provided for determining and/or classifying PIs and other skin injuries (or wounds) of a patient's tissue. For a non-invasive determination of PIs, an optical signal may be transmitted onto the patient's skin (e.g. by illuminating the skin) such that the reflected signal may be analyzed and/or monitored.

The optical radiation signals in different wavelengths that are transmitted and/or reflected through the living tissue of a suspected location of the patient's tissue, may be utilized for detection and/or measurement of changes in biomarkers that are associated with pressure injuries (e.g., stage 1 PIs), deep-tissue PIs, diabetic foot ulcers and other inflammatory skin injuries. Thus, a decision regarding an early-stage PI may be reached based on the changes or levels of the biomarkers.

The biomarkers for tissue injury, blood and tissue parameters may include at least one of: concentration of blood (e.g., red blood cells (RBC) concentration), oxygenated/de-oxygenated of Hemoglobin, blood flow regime, blood perfusion, arterial hemodynamic blood flow, capillary hemodynamic blood flow, endothelial hemodynamic blood flow, skin moisture, skin hydration, skin color, melanin concentration, viscosity, heart pulse wave propagation through tissue, skin palpitation (including non-blanching test), lipids concentration, skin ion capacitance or skin electrical conductivity, and local tissue temperature (e.g., measured by optical and/or mechanical thermometry). A combination of methods may be used to get a more precise tissue classification. Other skin conditions may include incontinence dermatitis classification, other skin dermatitis classifications, detection of skin graft implant level of blood supply during and after graft implant, detection of blood flow and blood perfusion changes in blood vessel closer to skin surface (e.g., to allow detection of blood clots during monitoring of carotid arteries).

For example, a Doppler effect or Speckle imaging may be employed for measuring blood flow and perfusion, utilizing a laser light source, where the signal is collected by detectors and the time dependency of the laser speckle characteristics is analyzed. The blood flow is measured at the capillary level (epidermis) for identifying stage 1 PI, and deeper close to the bone (dermis and subcutaneous layers) in order to identify DTI.

In some embodiments, a mobile device or system is provided (e.g., a hand-held device) for non-invasive monitoring of patient's tissue, where the monitoring may be carried out in real-time for early detection and treatment of tissue injuries, wounds and/or skin abnormalities. Such monitoring may provide a real time accurate diagnostic tool for decision support, having high accuracy regardless of skin tone or color, detecting incipient subdermal lesion of different types and accurately differentiating between numerous types of lesions.

Reference is made to FIG. 2, which is an illustration of a non-invasive device 200 for determination of a tissue injury, according to some embodiments of the invention.

The non-invasive device 200 may be an add-on attachment for a mobile computing device 20 (e.g., a hand-held mobile device) that is used by a patient or by a physician for determination of a tissue injury in the patient. The non-invasive device 200 may include a body having a shape that may correspond to any type of mobile computing device 20 (e.g., a smartphone), for example the body of the non-invasive device 200 may include an elastomeric (e.g., plastic or polymeric) portion 201 to be removably attached to a portion of the mobile computing device 20 that includes an imager (a smartphone camera 22) and/or a light source of the mobile computing device 20. By attachment of the non-invasive device 200 to the mobile computing device 20, it may be possible to carry out non-invasive measurements of patients while using commonly used hardware mobile devices (e.g., smartphones), as further described hereinafter.

In some embodiments, the elastomeric portion 201 may include an adjustable attachment sleeve like mechanism for universal removable attachment to any type of mobile computing device 20.

The non-invasive device 200 may include a power supply unit 202 (e.g., a rechargeable battery) that is configured to supply power to other elements of the non-invasive device 200. In some embodiments, the power supply unit 202 may receive electrical power from the battery of the mobile computing device 20.

For example, the power supply unit 202 may receive electrical power via wireless power charging (e.g., via an electromagnetic induction coil) from the battery of the mobile computing device 20. In another example, the power supply unit 202 may electrically connect to the power outlet of the mobile computing device 20 and receive electrical power directly for the non-invasive device 200.

According to some embodiments, the non-invasive device 200 may include an optical head 203 for sending optical signals towards the patient's skin and receiving optical signals reflected from the patient's skin.

The non-invasive device 200 may include an internal processor (e.g., such as the controller 105 shown in FIG. 1) for controlling functionality of the optical head 203, for instance via a dedicated mobile application. The internal processor may communicate with the processing unit of the mobile computing device 20, such that some hardware components of the mobile computing device 20 may be utilized for performing non-invasive measurements with the non-invasive device 200.

For example, the non-invasive device 200 may connect to the mobile computing device 20 such that the processor of the non-invasive device 200 may control functionality of the optical head 203 for non-invasive measurements as well as functionality of the mobile computing device 20, such as operation of the imager 22 and displaying on screen 23.

In some embodiments, an optical signal that is received at the optical head 203 may be the result of light being reflected from the patient's tissue in the visible light range, and/or in the infrared light range. Such reflection may be monitored as an indication of the tissue's condition of damage.

When pressure is applied on the skin (e.g., with a dedicated pressure sensor), one of the light diffusing elements in the human skin is the Elastin-Collagen complex which changes the 3-dimensional (3D) structure and thus changes the pattern of the back scattered light reflected from the tissue. Thus, by analyzing signals received at the optical head 203, it may be possible to determine no-pressure injury, stage 1 pressure injury or a deep tissue injury (DTI) based on the structural changes affecting the optical signal.

In some embodiments, the optical signal that is received at the optical head 203 (e.g., via an optical sensor) may be a light absorption, or a “fingerprint”, of the tissue including a concentration of molecules such as Melanin, Collagen-Elastin complex, fatty acids, Hemoglobin, oxy-hemoglobin, deoxy-hemoglobin. The different concentrations of these molecules at a given moment of measurement may be associated with a specific optical signature of the tested tissue such that identification of PIs may be achieved by monitoring of the optical signals.

When pressure is applied on the skin (e.g., with a dedicated pressure sensor), the concentration of these molecules may cause a change at the place of measurement (e.g., detectible by different wavelengths). As a pressure injury, or a skin condition, evolves, the concentration of these molecules in the tissue may change as well and the tissue reflection is changed accordingly.

In some embodiments, the optical signal that is received at the optical head 203 may be a dynamic signal that is associated with parameters as blood flow or as pulse wave propagation (or PWV pulse wave velocity) through the blood vessels of the patient's tissue in the measured area. When pressure is applied on the skin (e.g., with a dedicated pressure sensor), the oxygen supply may diminish and blood vessels thinner and larger may suffer from weak structure. If the damage or injury is in the upper layers of the skin (e.g., stage 1 injury) the thinner blood vessels may collapse, and if it is DTI the larger blood vessels may collapse since it is located deeper in the tissue.

In some embodiments, pulse oximetry may be employed with both a transmission pulse oximetry mode and a reflectance pulse oximetry mode, where the received signal is measured at one steady point along a given timeline. Spatial oximetry may be employed with both types of pulse oximetry, where may be measured in different places in the patient's tissue (e.g., in 3D matrices), in the same given time frame. For example, levels of oxygenation may be mapped in 3D in a given in a timeline for the patient's tissue.

Increased blood flow for example may be a sign of a potential superficial pressure injury due to inflammatory processes at the upper layers.

Reduced blood flow may be an indication of a deep tissue injury where a necrotic tissue may appear and diminished blood flow in the deeper blood vessels may demonstrate that these blood vessels are damaged. The same principle may be applied with PWV if there is a significant signal (or slightly diminished signal) when comparing suspected area to a normal area as a sign for a stage 1 pressure injury. If there is a drop in dynamic signal amplitude it may be a sign of deeper injury such as DTI.

In some embodiments, the optical head 203 may include at least one of: a pressure gauge, an electronic circuit with light sources and photo detectors, and an elastomeric cover.

According to some embodiments, the non-invasive device 200 may include a disposable tip 204 to be attached to the optical head 203 when performing a measurement of patient's tissue. The disposable tip 204 may include a polymeric frame with a multi snap design to allow firm attachment to the optical head 203 with snaps locked in a dedicated slit on the optical head 203. This design may allow easy removal of the disposable tip 204 from the optical head 203 by manually pulling off the disposable tip 204 or by pushing it off the optical tip 203 by widening the snaps contact with the optical head 203.

The disposable tip 204 may be at least partially transparent so as to allow light signals going through and back of the disposable tip 204 with no interference. In some embodiments, the disposable tip 204 may include barrier portions (e.g., a black polymeric plate with substantially small holes that is positioned above the light sources and sensors circuit) to prevent light reaching some of the optical sensors and/or to eliminate cross-talk between light sources and sensors.

In some embodiments, the optical head 203 may sense presence of a new disposable tip 204 on the non-invasive device 200 (e.g., by illuminating a coating of the disposable tip 204 and verifying that the expected signal is returned, or by scanning a dedicated barcode). It may be possible to remove the disposable tip 204 without touching the disposable tip itself by moving a small button (or lever) on the non-invasive device 200 thereby pushing the disposable off the device.

In some embodiments, the optical head 203 may be configured to capture a visual picture of site of measurement on patient's tissue with an autofocus camera CCD or other imager, for example using the camera of the mobile computing device 20. The camera may be controlled by the non-invasive device 200 (e.g., to focus on a particular area). The optical head 203 may also capture a thermal picture at site of measurement with a dedicated camera.

In some embodiments, the non-invasive device 200 may scan a suspected area similarly to an ultrasound hand scanner being able to calculate volume of damaged tissue, and determine a 3D shape of the damaged area. The non-invasive device 200 may determine the surface and/or depth of open wounds by image processing.

The non-invasive device 200 may include a communication unit 205 for wireless communication with the mobile computing device 20 and/or with other devices. For example, the communication unit 205 may communicate via wireless networks such as Bluetooth (via a dedicated Bluetooth Low Energy (BLE) unit), Wi-Fi, or other networks.

For taking a high-quality measurement from the patient's skin, it's advantageous to verify that the best predefined conditions are met before taking the actual measurement. The predefined list may be derived from the optical conditions and/or the environmental conditions (e.g., the level of pressure on patient's skin, the level of vibration of patient's body and/or nurse's hand holding the device, the ambient light in patient's room). According to some embodiment, to verify the predefined conditions before taking a measurement, a dedicated operation profile may be employed in the measurement device that exercises the light sources and photodetectors to obtain a continues optical signal, while continuously analyzing and processing the signal by an optimization algorithm that finds whether the prerequisite conditions are met or not. Once the predefined conditions are met, an automatic measurement may be taken from the patient's skin.

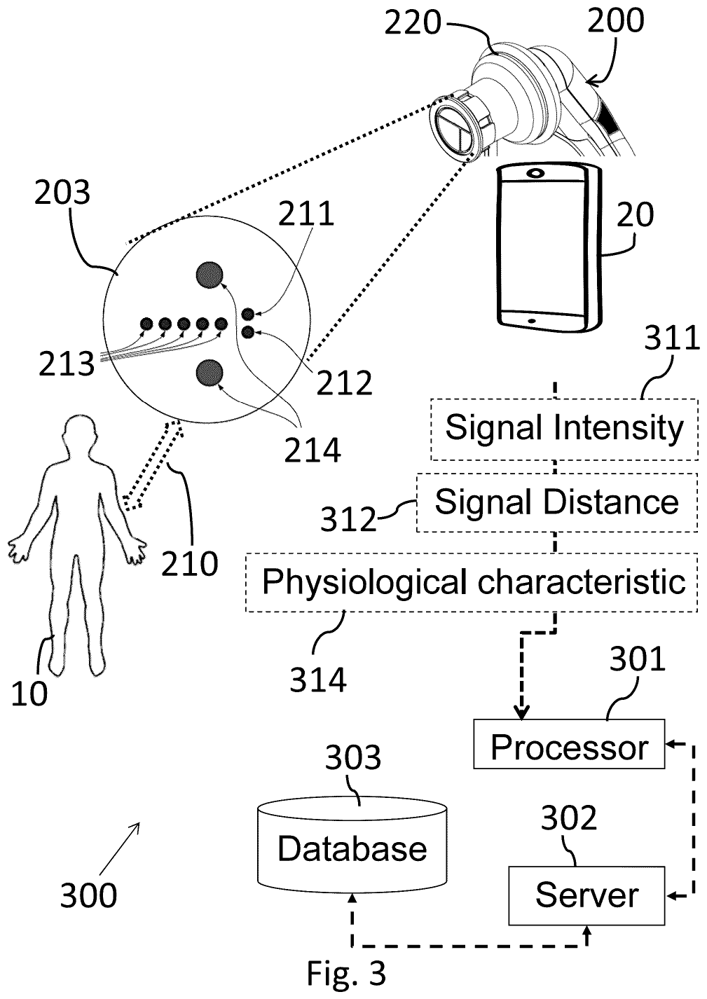

Reference is made to FIG. 3, which is an illustration of a non-invasive system 300 for determination of a tissue injury of a patient's tissue 10, according to some embodiments of the invention. Some elements in FIG. 3 are identical to corresponding elements in FIG. 2 having the same numerals, such as the non-invasive device 200 and the optical head 203.

In FIG. 3, the optical head 203 of the non-invasive device 200 is schematically shown as an enlarged portion that is adjacent to a patient's tissue 10. For example, the diameter of the optical head 203 may be in the range of 12-25 millimeters.

In some embodiments, the optical head 203 of the non-invasive device 200 may include a light source 211, 212. For example, the light source may be a single laser 211 in the near Infra-Red (NIR) range, or a multi-light source 212 in the visual and/or NIR and/or short-wave Infra-Red (SWIR) range. In some embodiments, at least one light source may be the light source of the mobile computing device 20.

The optical head 203 may further include at least two optical sensors 213. For example, the at least two optical sensors 213 may be an array of optical detectors (e.g., LEDs) in the visual and/or NIR and/or SWIR range. For example, the length of the array of optical detectors may be in the range of 2-15 millimeters.

For example, an amplitude and/or intensity of the received signal 210 may be detected by the at least two optical sensors 213.

The non-invasive device 200 may transmit different wavelengths of light as an optical signal 210 in the visual wave range and/or in the NIR range (400-1700 nanometers) into the patient's skin, with dedicated photodetectors placed in predetermined proximity to light sources in order to collect the reflected light signal 210. For example, light may be emitted wavelengths within the spectrum of 500 nanometer to 1700 nanometer, with at least one measurement in each of the following ranges: 500-600 nm, 600-700 nm, 700-800 nm, 800-900 nm, 900-1100 nm and 1200-1700 nm.

The at least two optical sensors 213 may receive data for signal intensity 311 and/or signal distance information 312 from light reflected from the tissue over at least two points of the patient's skin 10. In some embodiments, the signal distance information 312 may be measured between the light source 211, 212 and the at least two optical sensors 213. For example, for an array of at least two optical sensors 213 the light reflected from the tissue may cause optical reading in each of the at least two optical sensors 213, so that the signal distance information 312 may be associated with the distance between the light source 211, 212 and the corresponding optical sensor 213. For some injuries, a specific optical sensor 213 may provide results with higher accuracy so that the signal distance information 312 may be utilized to optimize the injury determination.

The data for signal intensity 311 and/or signal distance information 312 that is received by the at least two optical sensors 213 may be collected at a processor 301 (e.g., such as the controller 105 shown in FIG. 1) of the non-invasive device 200.

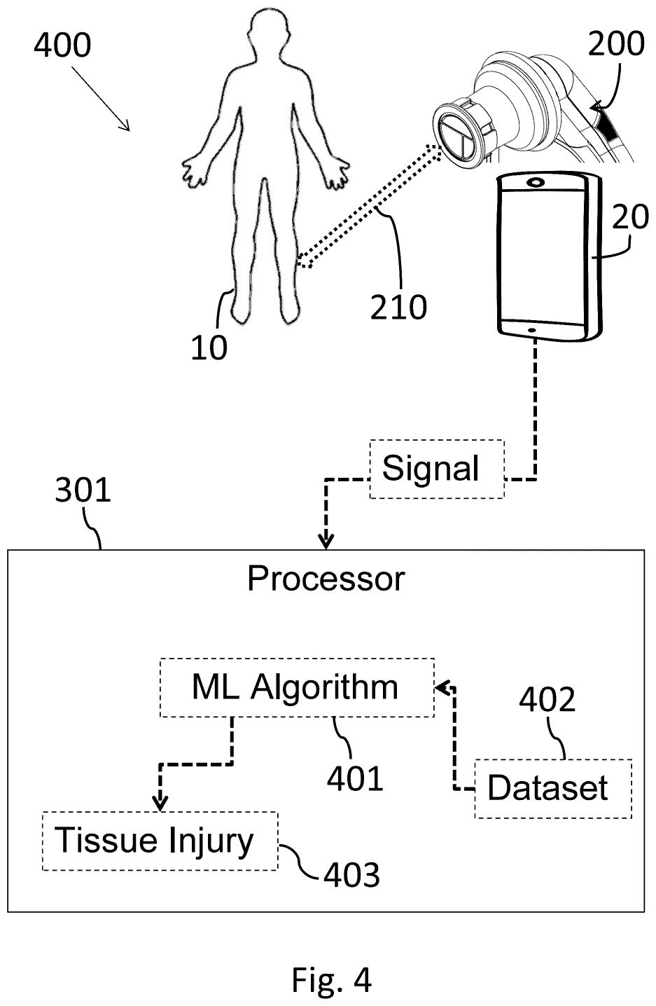

In some embodiments, the processor 301 shown in FIG. 3 and FIG. 4 may be the internal processor of the non-invasive device 200 or the processor of the mobile computing device 20.

The signal 210 may be received from a predetermined layer under the patient's intact skin 10, where the depth of the predetermined layer may correspond to the distance between the light source(s) and the optical sensors 213.

The optical head 203 may include a non-removable glass cover to keep optical elements undamaged during measurements as well as allow the glass to be cleanable.

In some embodiments, the optical head 203 of the non-invasive device 200 may include at least one physiological sensor 214, to measure a physiological characteristic information 314 of the tissue over at least two points of the patient's skin 10 (e.g., measure temperature of the patient's tissue 10 by a dedicated metal portion to transfer heat). The at least one physiological sensor 214 may be a sensor (e.g., a blood pulse sensor) to measure physiological characteristics of the patient, such as blood flow rate or pattern, temperature, hemoglobin level, and spatial oxygenation. For example, the temperature may be measured with a thermocouple or thermistors in direct contact to the skin, or bolometer in the range of 8-16 micron wavelength.

The physiological characteristic information 314 may be selected from at least one of: blood flow pattern, blood flow rate, blood viscosity, tissue temperature, skin tissue capacitance, pulse wave velocity, skin elasticity, hemoglobin level, and spatial oxygenation. For example, the physiological characteristic information 314 may be determined based on detection of myoglobin molecules in the patient's tissue 10 to indicate a deep tissue injury.

The processor 301 may receive signals representing structure differences in the patient's tissue layers and the corresponding biomarkers levels, calculating or determining relative changes in them, such as hemoglobin level (Oxy, Deoxy, and total), water content in tissue (for 970 and 1180 nanometers wavelengths are from a vibrational overtone of the O—H bond of water), and blood flow/perfusion, melanin level, Lipids (for 920, 1040, and 1210 nanometers wavelengths are associated with overtones of the stretching vibrational mode of the C—H bond). For example, absorption peak near a 1430 nm may be attributed to the first overtone of O—H stretching. Measuring temperature may also be considered when making a decision whether the tested tissue has an early stage pressure injury, and if so, also the type of the injury: stage 1, DTI, a non-pressure injury erythema, or no injury at all.

In some embodiments, the at least two points of the patient's skin 10 may be selected such that at least one point is determined as healthy tissue and at least one other point is determined as a potential subdermal injury. The non-invasive device 200 may accordingly measure a reference point on a healthy tissue of the patient 10, in order to get a normalized personalized result for each patient.

The optical data may be collected from different layers in different depths under intact skin of the patient 10 by using a number of light wavelengths in the visual and/or near infrared light bands. This combination of tissue parameters collection allows adaptation of the readings of the non-invasive device 200 to each patient's unique skin structure and/or tone (thereby achieving “personalized medicine”).

In order to achieve personalized profile for the patient 10, the non-invasive device 200 may perform calibration to the patient's skin type regardless to skin tone (to achieve personalization of the non-invasive device 200 to the patient 10). The calibration may be carried out by performing an optical scan of the patient's skin at different depths of the tissue by simultaneously using a number of wavebands of light ranging from the visual light to the NIR band, and taking a reference point at the “normal” or healthy skin area on the patient's body 10 and measure the “suspected” skin area where an injury may occur.

A reference point or location is a spot near the suspected area that rarely has pressure injury in it but may have similar tissue structure and other tissue properties. By measuring the relative changes in the biomarker levels between the suspected area and the reference area, it may be possible to identify the differences between the suspected injured area vs. the known to be healthy area and mitigate influences such as the natural skin tone of the same patient and other tissue's physiology that may be unique to the patient and influence the measurements.

By using the IR/NIR light band, improved signal to noise ratio may be achieved in darker skin tones regardless of different skin tones and/or colors. The non-invasive device 200 may analyze the comparison of absorbed and reflected light signal 210 from measurements as indication of the possibility of subdermal skin injury and its type, or different skin conditions as dermatitis or the ability to determine the blood perfusion.

By taking at least two measurements the non-invasive device 200 may become personalized to each patient's skin by measuring relative changes in tissue light absorbance and reflectance and blood flow dynamic parameters in various areas of the patient's body. Each patient has its own typical skin structure because of different skin tones, due to different skin layers compositions and skin thickness differences, different blood vessels spreading in the tissue, different hydration levels of the skin, etc. These skin parameters dramatically influence photons reflection and absorption through the skin so that personalized results for each patient 10 may substantially improve accuracy of the measurement.

In some embodiments, by determining the relative changes in acquired signals and performing the measurement consecutive for time intervals may allow to detect at least one of: incipient injuries, invisibles injuries, monitor developing processes under the skin in the dermis as well as the subdermal layers, identify changes that occur underneath the skin which may not be visible to the naked eye and allow earlier detection of a subdermal wound formation before a caregiver with a visible detection on the skin.

By taking at least two measurements the non-invasive device 200 in a patient's body location from two adjacent sites at substantially the same time, and by normalizing these signals 210 may allow the abnormalities which represent PIs to outstand from the rest of the signal and accordingly accurately identify tissue injuries.

In some embodiments, the collected optical data from the tested patient's skin areas may be combined or aggregated with one or more of additional dynamic parameters collected by the non-invasive device 200 such as: skin temperature, blood flow patterns, blood flow rate, blood viscosity, skin tissue capacitance, pulse wave velocity (PWV), skin elasticity measured ultrasonically by palpitation, total Hemoglobin level, and oxy/deoxy Hemoglobin measurement.

Another way to perform measurements by the optical head 203 is to apply the device only to areas in the suspected area (without a healthy reference) and to cross compare them to find the exception spot toward pressure ulcer. For example, using a symmetric body location for comparison (e.g., left heel and right heel).

In some embodiments, the processor 301 may be coupled to the at least two optical sensors 213 and the at least one physiological sensor 214 such that the information from these sensors may be received at the processor 301. Thus, the processor 301 may receive the data for signal intensity 311 and/or signal distance information 312 and/or physiological characteristic information 314.

The processor 301 may be embedded to the non-invasive device 200 such that the data from the at least two optical sensors 213 may directly pass to the processor 301. In some embodiments, processor 301 may be external to the non-invasive device 200, such that the data from the at least two optical sensors 213 may be transmitted (e.g., wirelessly) to the processor 301 for further processing.

The processor 301 may be coupled to a server 302, such that data received at the processor 301 may pass to the server 302 for further processing. For example, the server 302 may be coupled to dedicated database 303 for electronic medical records (EMR) or other physiological parameters such that analysis of data received at the processor 301 may be carried out at the server 302 (e.g., with dedicated computing components) based on information retrieved from the database 303.

For example, the processor 301 may provide a trajectory for patient pressure injury condition based on measurements history by the non-invasive device 200, as well as based on medical records (e.g., blood albumin level, blood CRP, blood glucose level, pressure injury history etc.).

According to some embodiments, the non-invasive device 200 may include an internal pressure mechanism or pressure sensor 220 for maintaining constant pressure on the patient's skin 10 during measurements by different operators so as to align the optical head 203 against the patient's skin surface and allow optimal contact with the patient's skin 10. The pressure sensor 220 may include a pressure gauge and/or a damping pressure mechanism (e.g., located above the pressure gauge in the shape of an elastomeric ring) to ensure fixed pressure that is applied during the measurement. For example, the pressure sensor 220 may initiate a measurement by the non-invasive device 200 when a pressure signal, from the pressure sensor 220, exceeds a predefined pressure threshold or is within a threshold range. In some embodiments, the damping pressure mechanism is separate from the pressure sensor as an independent unit.

The predetermined pressure threshold may be obtained by pressing the optical head 203 against the patient's skin at various time intervals. As the pressure threshold is being reached, the optical head 203 may release a series of lights beams (e.g., in a predetermined wavelengths of light in the visual and NIR light bands) emitted from the light source 211, 212. The series of light beams may be transmitted into the patient's skin and the back reflected light photons may be collected by the at least two light sensors 213.

In some embodiments, the pressure damping with a skin alignment mechanism may include an elastomeric ring or spring that holds the optical head 203. The pressure gauge may be positioned below the electronic circuit with light sources and/or sensors in order to sense the pressure applied on the optical head 203 when engaging the patient's skin 1. As the optical head 203 surface is pressed against the patient's skin it may adjust its upper surface of the optical head 203 to the patient's skin curvature to be in parallel with the skin surface due to the elasticity of the elastomeric ring. While pressing against the patient's skin, the pressure gauge under the optic probe may measure the pressure applied on the patient's skin.

In some embodiments, in order to accelerate the scanning time by the optical head 203, and also to increase the number of measurements, the non-invasive device 200 may utilized a modulation method, where each light source may be modulated simultaneously with a different frequency and utilizing algorithms such as Fast Fourier Transform (FFT), Raman transform, or “Locked In Amplifier” to extract each spectral component, as well as superheterodyne techniques.

In some embodiments, a spatial diffused-reflection light distribution may be determined to distinguish between healthy and pressure injury condition, for instance based on decay length of the signal. In some embodiments, the pulse waveform may be analyzed (e.g., using Photo-plethysmography (PPG)) in different to distinguish between healthy and a pressure injury condition.

According to some embodiments, by taking one measurement on the patient's skin, assuming all devices are calibrated and homogenous, and comparing between at least two different wavelengths by the light source 211, 212 with different response from the optical sensors 213 of this measurement. This comparison patterns may result in introducing changes in tissue light absorbance and/or reflectance from the patient's body or tissue. This comparison patterns may accordingly be used as input into the machine learning model that is trained on patient data and capable of inference of an accurate classification of the skin as healthy tissue, PI, stage 1 PI or DTI.

Reference is made to FIG. 4, which is an illustration of a non-invasive system 400 for determination of a tissue injury 403 with a machine learning (ML) algorithm 401, according to some embodiments of the invention. Some elements in FIG. 4 are identical to corresponding elements in FIG. 3 having the same numerals, such as the non-invasive device 200 and the processor 301.

In some embodiments, the processor 301 may train a machine learning (ML) algorithm 401 to determine a tissue injury 403 based on information received from the non-invasive device 200. The training may be carried out on a dataset 402 of intensity and signal distance information and physiological characteristic information of the tissue with signals 210 that are reflected from the patient's tissue 10 and received by sensors of the non-invasive device 200.

In some embodiments, the ML algorithm 401 may include ML classifiers for real-time, precise patient measurement as required for patient's skin assessment and/or examination and/or test (instead of monitoring the patient). The ML algorithm 401 may include a ML classifier such as a logistic regression model. The logistic regression model may determine the relationships between two data factors, and then use this relationship to predict the value of one of those factors based on the other. The ML algorithm 401 may also include ML classifiers such as neural networks.

Neural networks (NN) or connectionist systems are computing systems inspired by biological computing systems, but operating using manufactured digital computing technology. NNs are made up of computing units typically called neurons (which are artificial neurons or nodes, as opposed to biological neurons) communicating with each other via connections, links or edges. In common NN implementations, the signal at the link between artificial neurons or nodes can be for example a real number, and the output of each neuron or node can be computed by function of the (typically weighted) sum of its inputs, such as a rectified linear unit (ReLU) function. NN links or edges typically have a weight that adjusts as learning proceeds. The weight increases or decreases the strength of the signal at a connection. Typically, NN neurons or nodes are divided or arranged into layers, where different layers can perform different kinds of transformations on their inputs and can have different patterns of connections with other layers.

NN systems can learn to perform tasks by considering example input data, generally without being programmed with any task-specific rules, being presented with the correct output for the data, and self-correcting, or learning.

Various types of NNs exist. For example, a convolutional neural network (CNN) can be a deep, feed-forward network, which includes one or more convolutional layers, fully connected layers, and/or pooling layers. CNNs are particularly useful for visual applications. Other NNs can include for example transformer NNs, useful for speech or natural language applications, and long short-term memory (LSTM) networks.

In practice, a NN, or NN learning, can be simulated by one or more computing nodes or cores, such as generic central processing units (CPUs, e.g. as embodied in personal computers) or graphics processing units (GPUs such as provided by Nvidia Corporation), which can be connected by a data network. A NN can be modelled as an abstract mathematical object and translated physically to CPU or GPU as for example a sequence of matrix operations where entries in the matrix represent neurons (e.g. artificial neurons connected by edges or links) and matrix functions represent functions of the NN.

Typical NNs can require that nodes of one layer depend on the output of a previous layer as their inputs. Current systems typically proceed in a synchronous manner, first typically executing all (or substantially all) of the outputs of a prior layer to feed the outputs as inputs to the next layer. Each layer can be executed on a set of cores synchronously (or substantially synchronously), which can require a large amount of compute power, on the order of 10s or even 100s of Teraflops, or a large set of cores. On modern GPUs this can be done using 4,000-5,000 cores.

For example, the ML algorithm 401 may include live data normalization with live, data normalization in order to adapt to changing patient conditions and ensure precise measurement. In another example, the ML algorithm 401 may include skin contact identification with real-time detection of a proper skin contact that may enhance sensor accuracy as needed. In another example, the ML algorithm 401 may include dynamic preprocessing with imputation and/or scaling and/or feature selection for high-quality data, thereby supporting accurate patient assessments.

The machine learning algorithm 401 may be trained (e.g., with supervised training on labeled data items) on a set of light signals corresponding to various types of tissue injuries 403, such that a trained machine learning algorithm may predict presence of a tissue injury based on new signals measured by the non-invasive device 200.

After training of the machine learning algorithm 401, the processor 301 may apply the machine learning algorithm 401 on the received signal 210 to determine a subcutaneous tissue injury 403, for instance in which liquids accumulate subcutaneously. In some embodiments, the tissue injury 403 may be determined based on a calculated change in the received signal intensity 311, the signal distance information 312, and the physiological characteristic information 314 (e.g., as shown in FIG. 3).

For example, tissue classification based on measurements at two signals (e.g., in a reference point and a suspected measured point) of tissue parameters may include elevated temperature, or higher than reference point temperature as indication of stage 1 PI, also may be based on faster blood flow at suspected point than reference point, and may be based on a specific signal at point of measurement compared to reference point. The measurements for temperature or blood flow are not dependent on skin melanin concentration.

Another example is reduced temperature, or lower than reference point temperature as indication of DTI, also may be based on faster blood flow at reference point than in the suspected measured area, and may be based on a specific signal at point of measurement compared to reference point. The measurements for temperature or blood flow are not dependent on skin melanin concentration.

Another example is slightly temperature, or lower than reference point temperature as indication of non-pressure injury erythema (Dermatitis), also may be based on lightly faster of similar blood flow rate at suspected area compared to the reference point, and typical PI, and may be based on a specific signal at point of measurement compared to reference point. The measurements for temperature or blood flow are not dependent on skin melanin concentration.

Another example is no difference in temperature at reference point temperature as indication of tissue non PI condition with long lasting erythema (e.g., more than 10 minutes), also may be based on no difference in blood flow at reference point than in the suspected measured area, and may be based on a non specific signal at point of measurement compared to reference point. The measurements for temperature or blood flow are not dependent on skin melanin concentration.

In some embodiments, the processor 301 may calculate the probability of detection of an un open subdermal pressure injury (or diabetic foot ulcer), and/or the probability of differentiating between stage 1 PI, DTI, and other type of erythema by utilizing analysis different measured tissue parameters.

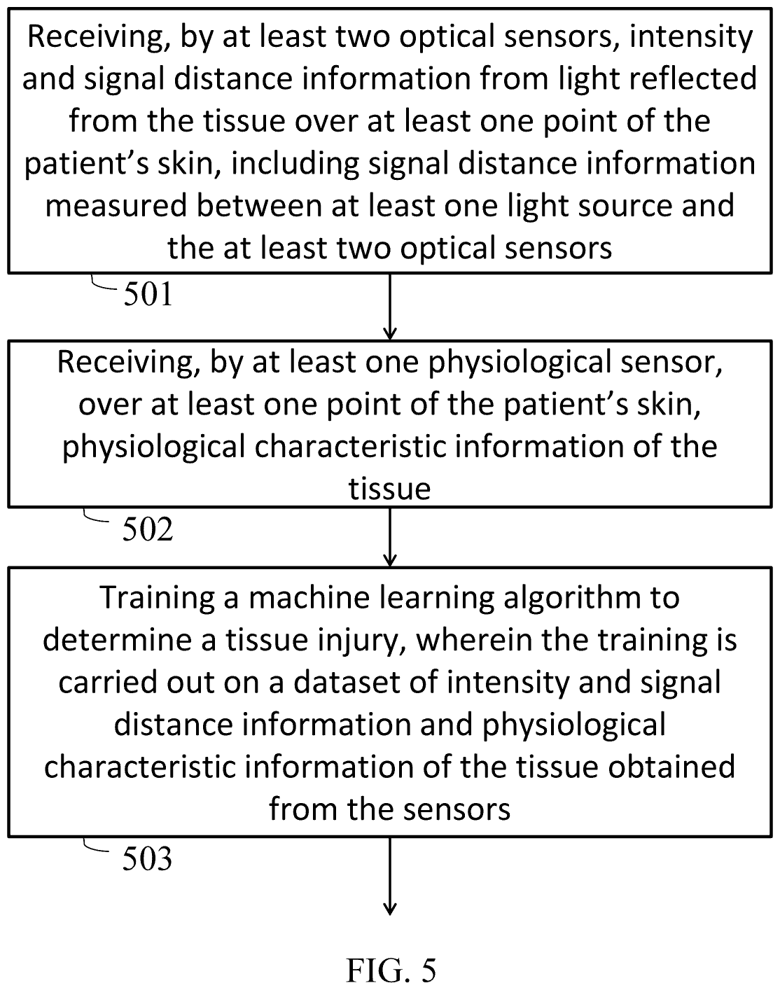

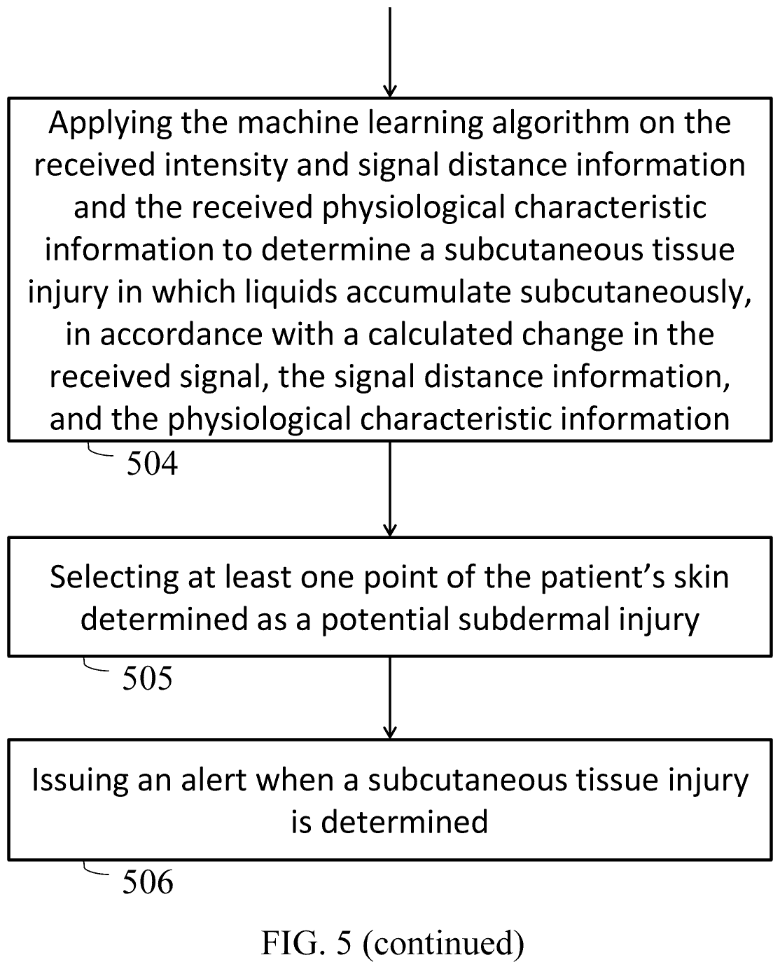

Reference is made to FIG. 5, which shows a flowchart for a method of non-invasive determination of a tissue injury, according to some embodiments of the invention.

In operation 501, intensity and signal distance information from light reflected from the tissue over at least two points of the patient's skin may be received. The signal distance information may be measured between at least one light source and the at least two optical sensors.

In operation 502, over at least two points of the patient's skin physiological characteristic information of the tissue may be received by at least one physiological sensor.

In operation 503, a machine learning algorithm may be trained by a processor to determine a tissue injury, wherein the training is carried out on a dataset of intensity and signal distance information and physiological characteristic information of the tissue obtained from the sensors.

In operation 504, the machine learning algorithm may be applied by the processor on the received signal to determine a subcutaneous tissue injury in which liquids accumulate subcutaneously, in accordance with a calculated change in the received signal, the signal distance information, and the physiological characteristic information.

In operation 505, at least one point of the patient's skin determined as a potential subdermal injury may be selected. In operation 506, an alert may be issued when a subcutaneous tissue injury is determined.

In some embodiments, the at least two points of the patient's skin are selected such that at least one point is determined as healthy tissue and at least one other point is determined as a potential subdermal injury.

While certain features of the invention have been illustrated and described herein, many modifications, substitutions, changes, and equivalents may occur to those skilled in the art. Various embodiments have been presented. Each of these embodiments may of course include features from other embodiments presented, and embodiments not specifically described may include various features described herein.

1. A method of non-invasive determination of a tissue injury of a patient's tissue, the method comprising:
attaching a non-invasive device to a mobile computing device;
receiving, by at least two optical sensors of the non-invasive device, intensity and signal distance information from light reflected from the tissue over at least one point of the patient's skin, including signal distance information measured between at least one light source and the at least two optical sensors;
receiving, by at least one physiological sensor, of the non-invasive device, over at least one point of the patient's skin, physiological characteristic information of the tissue;
training, by a processor of the mobile computing device, a machine learning (ML) algorithm to determine a tissue injury, wherein the training is carried out on a dataset of intensity and signal distance information and physiological characteristic information of the tissue obtained from the sensors;
applying, by the processor, the ML algorithm on the received intensity and signal distance information and the received physiological characteristic information to determine a subcutaneous tissue injury in which liquids accumulate subcutaneously, in accordance with a calculated change in the received signal intensity and signal distance information, and the physiological characteristic information;
selecting, by the processor, at least one point of the patient's skin determined as a potential subdermal injury; and
issuing, by the processor, an alert when a subcutaneous tissue injury is determined.

2. The method of claim 1, wherein at least one other than the point determined as a potential subdermal injury is determined as healthy tissue by the processor.

3. The method of claim 1, further comprising applying modulated lighting so as to accelerate the measurement time by the at least two optical sensors.

4. The method of claim 3, wherein the modulated lighting comprises using a different frequency for different light sources simultaneously based on a Fast Fourier Transform algorithm.

5. The method of claim 1, wherein the signal is received from the at least two optical sensors in a plurality of wavelengths.

6. The method of claim 1, wherein the physiological characteristic information of the tissue is selected from the group consisting of: blood flow pattern, blood flow rate, blood viscosity, tissue temperature, skin tissue capacitance, pulse wave velocity, skin elasticity, hemoglobin level, and spatial oxygenation.

7. The method of claim 1, wherein the physiological characteristic information is determined based on detection of myoglobin in the tissue.

8. The method of claim 1, further comprising initiating a measurement when pressure signal, from a pressure sensor, is within a predefined pressure threshold range.

9. The method of claim 8, wherein the pressure sensor is accommodated in an elastomeric ring.

10. The method of claim 1, further comprising measuring a reference point on a healthy tissue of the patient, in order to get a normalized personalized result for the patient.

11. A system for non-invasive determination of a tissue injury, of a patient's tissue, the system comprising:
a light source;
at least two optical sensors, configured to receive intensity and signal distance information from light reflected from the tissue over at least one point of the patient's skin, including signal distance information measured between the light source and the at least two optical sensors;
at least one physiological sensor, configured to receive over at least one point of the patient's skin physiological characteristic information of the tissue;
a processor of a mobile computing device, coupled to the at least two optical sensors and the at least one physiological sensor, wherein the processor is configured to:
train a machine learning (ML) algorithm to determine a tissue injury, wherein the training is carried out on a dataset of intensity and signal distance information and physiological information of the tissue obtained from the sensors;
apply the ML algorithm on the intensity and signal distance information obtained from the optical sensors to determine a subcutaneous tissue injury in which liquids accumulate subcutaneously, in accordance with a calculated change in the received signal intensity and signal distance information, and the physiological characteristic information, and display the determination;
select at least one point of the patient's skin determined as a potential subdermal injury; and
issue an alert when a subcutaneous tissue injury is determined.

12. The system of claim 11, wherein a signal is received from the at least two optical sensors in a plurality of wavelengths.

13. The system of claim 11, wherein the physiological characteristic information of the tissue is selected from the group consisting of: blood flow pattern, blood flow rate, blood viscosity, temperature, skin tissue capacitance, pulse wave velocity, skin elasticity, hemoglobin level in tissue, hemoglobin saturation rate, and spatial oxygenation level.

14. The system of claim 11, wherein the physiological characteristic information is determined based on detection of myoglobin in the tissue.

15. The system of claim 11, wherein the processor is to initiate a measurement when pressure signal, from a pressure sensor, is within a predefined pressure threshold range.

16. The system of claim 11, wherein the processor is to initiate a measurement when pressure signal, from a damping pressure mechanism, is within a predefined pressure threshold range.

17. The system of claim 11, wherein the at least two optical sensors are to measure a reference point on a healthy tissue of the patient, in order to get a normalized personalized result for the patient.

18. A device for non-invasive determination of a tissue injury of a patient's tissue, the device comprising:
a light source;
at least two optical sensors, configured to receive intensity and signal distance information from light reflected from the tissue over at least one point of the patient's skin, including signal distance information measured between the light source and the at least two optical sensors;
at least one physiological sensor, configured to receive over at least one point of the patient's skin physiological characteristic information of the tissue;
a processor of a mobile computing device, coupled to the at least two optical sensors and the at least one physiological sensor, wherein the processor is configured to:
train a machine learning (ML) algorithm to determine a tissue injury, wherein the training is carried out on a dataset of intensity and signal distance information and physiological information of the tissue obtained from the sensors;
apply the ML algorithm on the intensity and signal distance information and physiological information obtained from the optical sensors to determine a subcutaneous tissue injury in which liquids accumulate subcutaneously, in accordance with a calculated change in the received signal intensity and signal distance information, and the physiological characteristic information;
select at least one point of the patient's skin determined as a potential subdermal injury; and
issue an alert when a subcutaneous tissue injury is determined.
